SEQUENCE LISTING (3 sequences)

SEQ ID NO: 1 - protein, 53 aa, Homo sapiens
NSDSECPLSHDGYCLHDGVCMYIEALDKYACNCVVGYIGERCQYRDLKWWELR

SEQ ID NO: 2 - protein, 21 aa, Artificial Sequence
  EGF fragment
DGYCLHDGVCMYIEALDKYAC

SEQ ID NO: 3 - protein, 21 aa, Artificial Sequence
  EGF fragment
DGYCLHDGVSMYIEALDKYAC

(19) United States
(12) Patent Application Publication
(10) Pub. No.: US 2021/0355448 A1
(43) Pub. Date: Nov. 18, 2021

(54) METHODS OF ISLET CELL CULTURE

(71) Applicant: CITY OF HOPE, Duarte, CA (US)

(72) Inventors: Jose Enrique Montero CASIMIRO, Duarte, CA (US); Bart Otto ROEP, Duarte, CA (US); Ismail H. AL-ABDULLAH, Duarte, CA (US)

(21) Appl. No.: 16/625,501

(22) PCT Filed: Jun. 26, 2018
(86) PCT No.: PCT/US18/39601

§ 371 Date: Dec. 20, 2019

Related U.S. Application Data
(60) Provisional application No. 62/525,090, filed on Jun. 26, 2017.

(51) Int. Cl. C12N 5/071; A61K 38/18; A61K 35/39

(52) U.S. Cl. CPC C12N 5/0676; A61K 38/1808; A61K 35/39; C12N 2501/335; C12N 2500/34; C12N 2501/345; C12N 2501/11

(57) ABSTRACT

Provided herein are, inter alia, methods, compositions, and kits for culturing and improving the quality of islet cells. Included are compositions and kits for the ex vivo culture of islets or islet cells as well as methods of making and using the same. Isolated and cultured islets and islet cells are also provided.

### CROSS-REFERENCES TO RELATED APPLICATIONS

This application claims priority to U.S. Provisional Application No. 62/525,090, filed Jun. 26, 2017, which is hereby incorporated by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file “048440-669001WO_SEQUENCE_LISTING.txt”, created on Jun. 26, 2018, 1,328 bytes, machine format IBM-PC, MS-Windows operating system, is hereby incorporated by reference.

### BACKGROUND

Pancreatic islet quality is essential to warranty the in vivo efficacy thereof after transplantation, restoring the insulin production in diabetic patients. However, there is no effective and simple composition or method available to improve the islet cell quality. The compositions and methods provided herein address these and other needs in the art.

### BRIEF SUMMARY

Provided herein are, inter alia, methods, compositions, and kits for culturing and improving the quality of islet cells (e.g., the viability and/or the suitability thereof for administration to a subject in need thereof). Isolated and cultured islet cells are also included.

In an aspect, provided herein is a method of culturing a population of islet cells. In embodiments, the method includes culturing the population of islet cells in a culture medium having epidermal growth factor (EGF) or a fragment thereof.

In an aspect, provided herein is a method of producing a cultured islet cell population. In embodiments, the method includes culturing an islet cell population in a culture medium that includes EGF or a fragment thereof, thereby forming the cultured islet cell population.

In an aspect, provided herein is a method of increasing the viability or function of an islet cell population. In embodiments, the method includes contacting the islet cell population with an effective amount of EGF or a fragment thereof.

In an aspect, a method of increasing the survival or recovery of islet cells isolated from islet tissue is provided. In embodiments, the method comprises contacting the islet cells with an effective amount of EGF or a fragment thereof.

In an aspect, included herein is an isolated islet cell that has been cultured ex vivo according to a method provided herein.

In an aspect, provided herein is an ex vivo culture. In embodiments, the ex vivo culture includes an islet cell population in a culture medium, wherein the culture medium comprises EGF or a fragment thereof. In embodiments, the islet cell population is a fully differentiated islet cell population.

In an aspect, included herein is a method of administering (e.g., transplanting) to a subject in need thereof an islet cell population. In embodiments, the islet cell population has been cultured ex vivo according to a method provided herein.

In an aspect, provided herein is a method of treating a disease in a subject in need thereof, wherein the disease is associated with reduced islet cell function or survival. In embodiments, the method includes administering to the subject an islet tissue or an islet cell population disclosed herein. In embodiments, the method includes administering EGF or a fragment thereof to the subject.

In an aspect, included herein is a cell culture medium, transplant storage medium, or kit for maintaining or culturing an islet cell population. In embodiments, the cell culture medium, transplant storage medium, or kit includes EGF or a fragment thereof.

### BRIEF DESCRIPTION OF THE DRAWINGS

FIG. 1. Images showing epidermal growth factor receptor (EGFR) and activated leukocyte cell adhesion molecule (ALCAM) immunofluorescence cell staining of human pancreas tissues. EGFR and ALCAM were visualized through green channel and insulin was visualized through red channel.

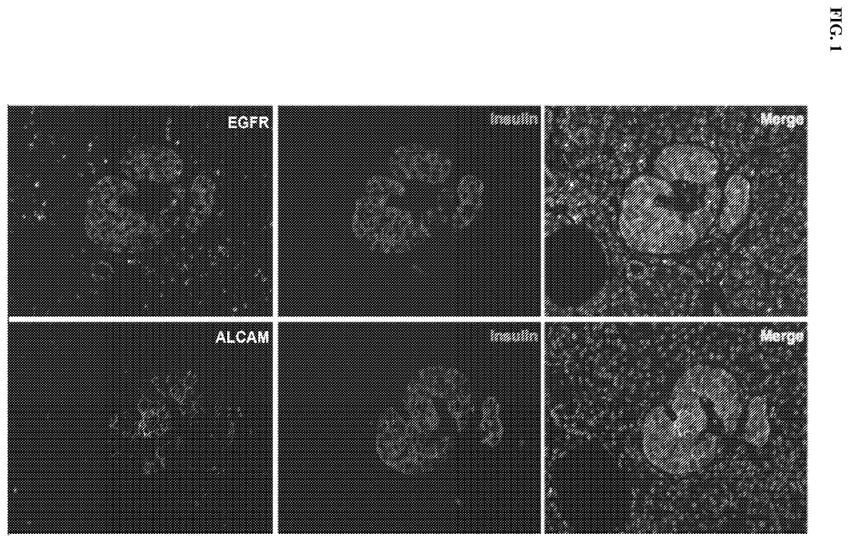

FIGS. 2A-2D. FIG. 2A. Timeline of the experiments illustrated in FIGS. 2B-2D. FIGS. 2B-2D. Glucose-stimulated increment in oxygen consumption rate (OCR) test using Seahorse Analyzers.

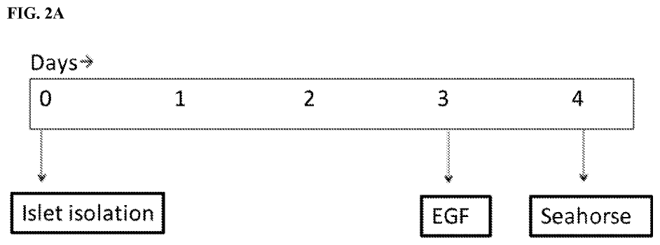

FIG. 3. A series of representative images of paraffin-embedded pancreatic tissue sections from a normal donor stained for EGFR-ECD and EGFR-ICD. Original magnification is 40×, taken on a Zeiss LSM 700 Confocal microscope.

FIG. 4. Graph showing cell death under the following conditions: (1) Media (Control); (2) high concentration (High Cyto) of human recombinant cytokines (1000 IU/ml IFN-γ, 1000 IU/ml TNF-α and 50 IU/ml IL-1β); (3) low concentrations of human recombinant cytokines (Low Cyto: 100 IU/ml IFN-γ, 100 IU/ml TNF-α and 5 IU/ml IL-1β); (4) supernatant (100%) of a β-cell antigen specific autoreactive T-cell clone isolated from a prediabetic donor and stimulated in vitro with islet peptide (T-cell SP); and (5) supernatant (100%) of a β-cell antigen specific autoreactive T-cell clone isolated from a prediabetic donor and stimulated in vitro with control peptide (T-cell NS). Approximately 70 islets/well of isolated human pancreatic islets were cultured in Seahorse XF24 islet-capture-microplates at 37° C. and 5% CO2 in 200 μl of CMRL islet culture media. Islets were cultured with or without 10 ng/ml of human recombinant EGF in all culture conditions. Cell death was measured after 24 h by incubating 2 μg/ml of Propidium Iodide (PI) for 15 min at room temperature, and scanned in the Celigo Image Cytometer. Two-way ANOVA, *p<0.05, T-cell SP p=0.0231, High Cyto p=0.0137, n=3. The EGF protective effect is observed in the range from 2 to 10 ng/ml.

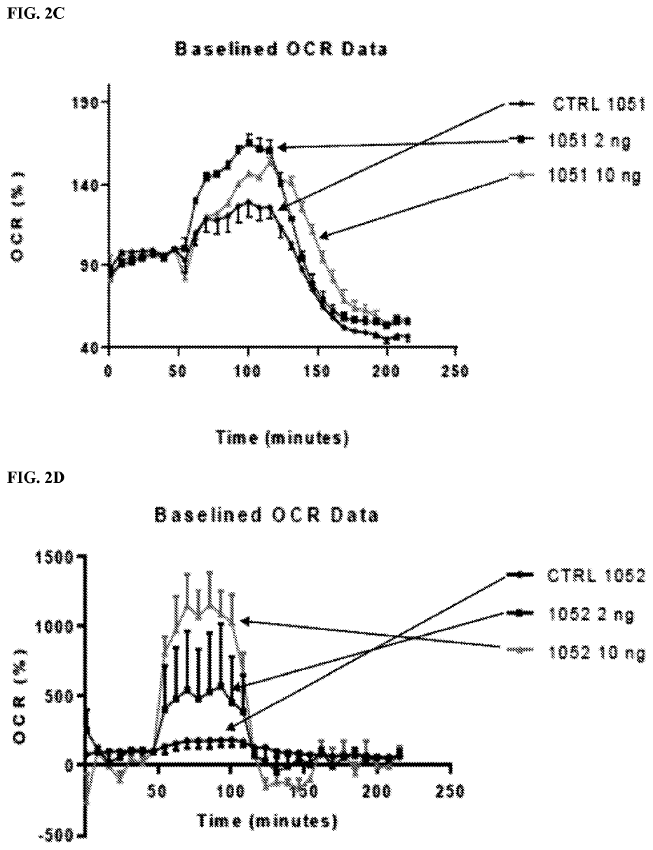

FIG. 5. Graph showing cell death rate of the human islets in culture compared to Media (Control). The following conditions were evaluated: (1) human recombinant EGF 10 ng/ml or (2) 100 ng/ml; (3) low concentrations (1×) of human recombinant cytokines (Low Cyto: 100 IU/ml IFN-γ, 100 IU/ml TNF-α and 5 IU/ml IL-1β); high concentration (10×) of human recombinant cytokines (High Cyto: 1000 IU/ml IFN-γ, 1000 IU/ml TNF-α and 50 IU/ml IL-1β) and Triton X-100, as positive control for cell death induction. Approximately 70 islets/well of isolated human pancreatic islets were cultured in Seahorse XF24 islet-capture-microplates at 37° C. and 5% CO2 in 200 μl of CMRL islet culture media. Cell death was measured after 24 h by incubating 2 μg/ml of Propidium Iodide (PI) for 15 min at room temperature, and scanned in the Celigo Image Cytometer.

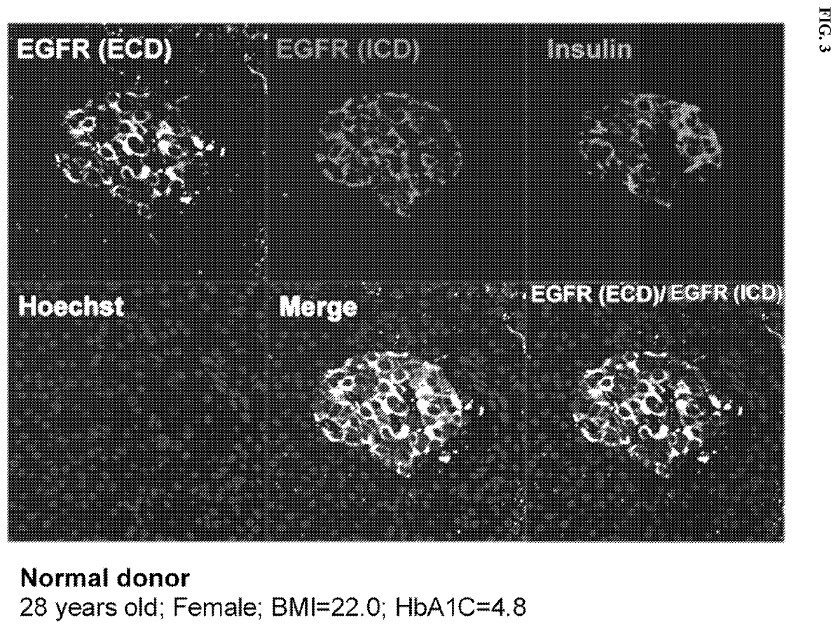

FIG. 6. Images of western blots of protein extracts from human islets from a normal donor treated with or without 10 ng/ml EGF and 50 units/mL IL-1β, 1,000 units/mL TNF-α, and 1,000 units/mL IFN-γ for 24 h.

FIG. 7. Graph showing relative cell death of the average of 3 human islets donors in culture under the following conditions: (1) Media (Control); (2) human recombinant EGF 10 ng/ml; (3) high concentration (Cytokines) of human recombinant cytokines (1000 IU/ml IFN-γ, 1000 IU/ml TNF-α and 50 IU/ml IL-1β) and both, (4) high concentration (Cytokines) of human recombinant cytokines (1000 IU/ml IFN-γ, 1000 IU/ml TNF-α and 50 IU/ml IL-1β) and the human recombinant at EGF 10 ng/ml. Approximately 70 islets/well of isolated human pancreatic islets were cultured in Seahorse XF24 islet-capture-microplates at 37° C. and 5% CO2 in 200 μl of either in CMRL or PIM(R) islet culture media. Cell death was measured after 24 h by incubating 2 μg/ml of Propidium Iodide (PI) for 15 min at room temperature, and scanned in the Celigo Image Cytometer. Two-way ANOVA with Bonferroni's post-hoc test. *p<0.05, **p<0.01. n=3.

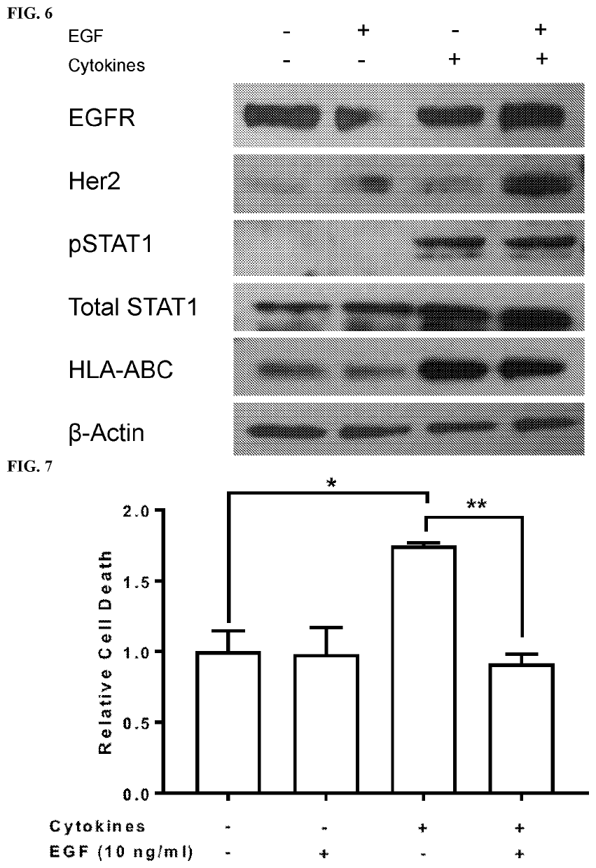

FIG. 8. Images of islet cells in capture wells. Approximately 70 islets/well of isolated human pancreatic islets are cultured in Seahorse XF24 islet-capture-microplates at 37° C. and 5% CO2 in 200 μl of either in CMRL or PIM(R) islet culture media. Look under microscope to check whether all islets are in capture well, otherwise islets can be shoved in with a pipet tip. C1 to C6 denote example of 6 different plate wells with different amount and distribution of islets.

### DETAILED DESCRIPTION

Provided herein are, inter alia, compositions (such as culture and transplant storage media) and methods for improving the viability of islets and islet cells. Included herein are compositions and methods that increase the quality of islets and islets cells that would otherwise be discarded rather than administered to subjects in need of islet treatment. Aspects relate to the achievement of improved yield and functionality of islet cells for transplantation. In embodiments, the number of islets or islet cells that can be isolated from a single donor is increased. In embodiments, less donor tissue is needed to treat a single subject.

Non-limiting and illustrative considerations and examples are as follows:
1. In aspects, methods and compositions provided herein overcome challenges regarding the availability and viability of islet cells. In embodiments, the duration of in vitro (e.g., ex vivo) islet cell treatment provides important advantages. A solution provided herein includes a short-term conditioning treatment with EGF that leads to a better yield and quality of an islet cell preparation for transplantation. In embodiments, the culture of islet cells in medium comprising EGF or a fragment thereof occurs during the first 72 hours after the collection of islet tissue to transplantation. In embodiments, this is during the “Manufacturing Islets Product” period prior to the inoculation into a subject (such as a recipient with Type 1 diabetes). During the islet cell isolation process, islet cells are subject to extreme stress conditions due to the use of enzymes, low pH and the auto-degradation of the pancreas during and immediately after the end-of-life period (e.g., when obtained from deceased donors). Medications, drugs, oxygen deficit, hypo or hyperglycemia, electrolytic imbalance, pancreatic enzymes, etc. affect the survival and quality of islet cells for transplantation. In embodiments, included herein is a solution where a short-term conditioning treatment with EGF leads to a better yield and quality of the islet preparation for transplantation. In embodiments, such an approach has advantages compared to culturing cells for long periods of time (e.g., chronically, such as for 4 weeks or more).
2. In aspects, the concentration of EGF or a fragment thereof offers important advantages. In embodiments, an effective amount of EFG (e.g., to increase islet cell viability, function or suitability for transplantation) is achieved at a concentration of EGF of 10 ng/ml. In embodiments, an effective amount of EGF concentrations is from 2 ng/mL to 10 ng/mL. In embodiments, the effective amount is also effective to reduce the immunogenicity of the islet cells.
3. In aspects, the glucose concentration in culture medium provides important advantages. In embodiments, islet cells are maintained (e.g., cultured) under physiological concentrations of glucose (e.g., 5.5 mmol/liter (mM)), reducing or preventing alteration of the insulin producing cells (beta cells) in the islets that latter will be subject for transplantation. Severe hyperglycemia (11 mM) can modify the normal physiology of beta cells, including the expression of gastrin. See, e.g., Dahan T, Ziv 0, Horwitz E, Zemmour H, Lavi J, Swisa A, Leibowitz G, Ashcroft F M, In't Veld P, Glaser B, Dor Y. Pancreatic β-Cells Express the Fetal Islet Hormone Gastrin in Rodent and Human Diabetes. Diabetes. 2017 February; 66(2):426-436, the entire content of which is incorporated herein by reference. In embodiments, the concentration of glucose remains unaltered at about 5.5 mmol/liter. In embodiments, such an approach has advantages compared to culturing cells under artificial a supra-physiological concentration of glucose of, e.g., 11 mmol/liter (mM).
4. In aspects, advantages relate to the type of donor that can be rescued for an effective transplantation. It has been shown a limited survival in vivo of islets after transplantation if coming from hyperglycemic donors. For that reason, it is a common practice to use only islets from donors with 5.6 (%) Hemoglobin A1c (HbA1c). Consequently, donors with HbA1c greater than 5.6% are precluded from use for transplantation. The procedure of short-term conditioning treatment with EGF has been evaluated in islets from donors with HbA1c ranging from 4.9 to 6.1% obtaining similar benefit (Table below).

| | | | | HbA1c | IEQ/ | Purity | Islet |
|---|---|---|---|---|---|---|---|
| Gender | Race | Age | BMI | (%) | IPN | (%) | Grade |
| M | Caucasian | 23 | 26.5 | 6.1 | 0.84 | 78 | B |
| M | Hispanic | 27 | 24.7 | 5.0 | 0.74 | 75 | B |
| F | Caucasian | 43 | 25.0 | 4.9 | 2.57 | 75 | A |

Thus, provided herein is a solution where a short-term conditioning treatment with EGF makes viable the use of islets from donors with HbA1c>5.6%, increasing the availability of material for transplantation.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. Any chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

The terms “a” or “an,” as used in herein mean one or more.

The transitional term “comprising,” which is synonymous with “including,” “containing,” or “characterized by,” is inclusive or open-ended and does not exclude additional, unrecited elements (such as method steps or ingredients). By contrast, the transitional phrase “consisting of” excludes any element not specified in the claim. The transitional phrase “consisting essentially of” limits the scope of a claim to the specified materials or steps “and those that do not materially affect the basic and novel characteristic(s)” of the claimed invention. Where methods and compositions are disclosed using the transitional term “comprising” it will be understood that corresponding methods and compositions with the transitional term “consisting of” and “consisting essentially of” are also disclosed.

Where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Depending on context, the term “biological sample” or “sample” refers to a material or materials obtained from or derived from a subject or patient. In embodiments, a biological sample includes sections of tissues such as biopsy and autopsy samples, and frozen sections taken for histological purposes. Non-limiting examples of samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In embodiments, a sample comprises islet cells. In embodiments, a sample comprises blood or another fluid from a subject to assess the progression of disease (e.g. diabetes), and/or to assess glucose levels in the subject. In embodiments, samples that have been obtained from the subject before and after a treatment are collected, and the level of glucose in the samples is measured to assess whether the subject's glucose levels have improved (e.g., are more similar to a normal control) after treatment compared to before treatment.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian (e.g., human) and insect (e.g., spodoptera) cells. Cells may be useful when they are naturally nonadherent or have been treated not to adhere to surfaces, for example by trypsinization.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or Fab domain) corresponds to light chain threonine at Kabat position 40, when the selected residue occupies the same essential spatial or other structural relationship as a light chain threonine at Kabat position 40. In embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant.” In embodiments, the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” In some embodiments, two sequences are 100% identical. In embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In embodiments, the identity exists over a region that is at about 10 nucleotides in length, or more preferably over a region that is 20 to 50, 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. In various embodiments, a comparison window is the entire length of one or both of two aligned sequences. In some embodiments, two sequences being compared comprise different lengths, and the comparison window is the entire length of the longer or the shorter of the two sequences. In certain embodiments relating to two sequences of different lengths, the comparison window includes the entire length of the shorter of the two sequences. In some embodiments relating to two sequences of different lengths, the comparison window includes the entire length of the longer of the two sequences. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,-2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

In embodiments, an indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid”, “vector” or “expression vector” refer to a nucleic acid molecule that encodes for a gene and/or regulatory elements necessary for the expression of a gene. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. In embodiments, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “ligand” refers to an agent, e.g., a polypeptide or other molecule, capable of binding to a receptor. In embodiments, a ligand is ligand capable of binding to an epidermal growth factor receptor (EGFR). In embodiments, a ligand is epidermal growth factor (EGF).

The term “EGF” as referred to herein includes any of the recombinant or naturally-occurring forms of EGF or fragments, variants or homologs thereof that maintain EGF activity (e.g. within at least 50%, 80%, 90%, 95%, 96%, 97%, 98%, 99% or 100% activity compared to EGF). In embodiments, the variants or homologs have at least 85%, 90%, 95%, 96%, 97%, 98%, 99% or 100% amino acid sequence identity across the whole sequence or a portion of the sequence (e.g. a 10, 20, 30, or 40 continuous amino acid portion) compared to a naturally occurring EGF protein. In embodiments, the EGF protein is substantially identical (e.g., at least 85%, 90%, 95%, 96%, 97%, 98%, 99% or 100% identical) to the amino acid sequence of SEQ ID NO: 1

(NSDSECPLSHDGYCLHDGVCMYIEALDKYACNCVVGYIGERCQYRDL
KWWELR).

The term “fragment,” as used herein, means a portion of a polypeptide or polynucleotide that is less than the entire polypeptide or polynucleotide. As used herein, a “functional fragment” of a protein, e.g., EGF, is a fragment of the polypeptide that is shorter than the full-length, immature, or mature polypeptide and has at least 25% (e.g., at least 30%, 40%, 50%, 60%, 70%, 80%, 90%, 95%, 98%, 99%, or even 100% or more) of the activity of full-length mature reference protein. Fragments of interest can be made by recombinant, synthetic, or proteolytic digestive methods. In embodiments, EGF fragments described herein have total lengths of about 50 , about 45, about 40, about 35, about 30, about 25, about 20, about 15, or about 10 amino acids in length (including all intermediate lengths). In embodiments, an EGF fragment includes an amino acid of SEQ ID NO: 2 (DGYCLHDGVCMYIEALDKYAC). In embodiments, an EGF fragment includes an amino acid of SEQ ID NO: 3 (DGYCLHDGVSMYIEALDKYAC [quasi-cyclic peptide (Cys4-Cys21) with a Cys-to-Ser substitution at position 10]).

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Patient” or “subject in need thereof” refers to a living member of the animal kingdom suffering from or that may suffer from the indicated disorder. In embodiments, the subject is a member of a species that includes individuals who naturally suffer from the disease. In embodiments, a subject is a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In embodiments, a patient is human.

The terms “disease” or “condition” refer to a state of being or health status of a patient or subject capable of being treated with a compound, transplant (such as an islet), cell (such as an islet cell), pharmaceutical composition, or method provided herein. In embodiments, the disease is an autoimmune disease. In embodiments, the disease is diabetes mellitus type 1.

An “autoimmune disease” as used herein refers to a disease or disorder that arises from altered immune reactions by the immune system of a subject, e.g., against substances tissues and/or cells normally present in the body of the subject. Autoimmune diseases include, but are not limited to, arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, Type 1 diabetes, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, and allergic asthma.

The term “associated” or “associated with” in the context of a substance or substance activity or function associated with a disease (e.g., Type 1 diabetes) means that the disease (e.g. Type 1 diabetes) is caused by (in whole or in part), or a symptom of the disease is caused by (in whole or in part) the substance or substance activity or function. In embodiments, a patient has a disease associated with reduced islet cell function or survival.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, “treating” refers to treatment of an autoimmune disease (e.g., Type 1 diabetes). In embodiments, the autoimmune disease is associated with reduced islet cell function or survival.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

In embodiments, the term “administering” includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. In embodiments, administration is by any route, including parenteral and transmucosal (e.g., buccal, sublingual, palatal, gingival, nasal, vaginal, rectal, or transdermal) routes. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

Pharmaceutical compositions may include compositions wherein the active ingredient (e.g. a compound, protein such as EGF, cell such as an islet cell, or an islet described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., increasing survival or suitability of a transplant or cell, modulating the activity of a target molecule, and/or reducing, eliminating, or slowing the progression of disease symptoms.

The term “islets,” “pancreatic islets,” or “islets of Langerhans” as used herein refers to the regions of the pancreas that contain its endocrine (i.e., hormone-producing) cells. The pancreatic islets are arranged in density routes throughout the human pancreas, and are important in the metabolism of glucose. There are about 3 million islets distributed in the form of density routes throughout the pancreas of a healthy adult human, each of which measures an average of about 0.1 mm (109 μm) in diameter. Each is separated from the surrounding pancreatic tissue by a thin fibrous connective tissue capsule which is continuous with the fibrous connective tissue that is interwoven throughout the rest of the pancreas. The combined mass of the islets is about 2 grams. There are five types of cells in an islet: alpha cells that make glucagon, which raises the level of glucose (sugar) in the blood; beta cells that make insulin; delta cells that make somatostatin which inhibits the release of numerous other hormones in the body; and PP cells and D1 cells, about which little is known. Degeneration of the insulin-producing beta cells is the main cause of type I (insulin-dependent) diabetes mellitus.

In embodiments, islet cells are cells originated from an islet. In embodiments, islet cells are within an islet. In embodiments, islet cells include alpha cells, beta cells, delta cells or any mixture or combination thereof. In embodiments, the islet cells are alpha cells. In embodiments, the islet cells are beta cells. In embodiments, the islet cells are delta cells.

Alpha cells (more commonly alpha-cells or α-cells) are endocrine cells in the pancreatic islets of the pancreas. They make up to 20% of the human islet cells synthesizing and secreting the peptide hormone glucagon, which elevates the glucose levels in the blood. When being viewed by an electron microscope, alpha cells can be identified by their characteristic granules with a large dense core and a small white halo.

Beta cells (β cells) are a type of cell found in the pancreatic islets of the pancreas. They make up 65-80% of the cells in the islets. The primary function of a beta cell is to store and release insulin. Beta cells can respond quickly to spikes in blood glucose concentrations by secreting some of their stored insulin while simultaneously producing more.

Delta cells (δ-cells or D cells) are somatostatin-producing cells. They can be found in the stomach, intestine and the pancreatic islets. In rodents, delta-cells are located in the periphery of the islets; in humans the islet architecture is generally less organized and delta-cells are frequently observed inside the islets as well. In both species, the peptide hormone Urocortin3 (Ucn3) is a major local signal that is released from beta cells (and alpha cells in primates) to induce the local secretion of somatostatin. Viewed under an electron microscope, delta-cells can be identified as cells with smaller and slightly more compact granules than beta cells.

In embodiments, the bioactivity or the function of islet cells refers to (or is assessed by) the level of peptide hormone released by the cells (e.g., glucagon by alpha cells, insulin by beta cells and somatostatin or Ucn3 by delta cells).

Pancreatic progenitor cells are multipotent stem cells originating from the developing fore-gut endoderm which have the ability to differentiate into the lineage specific progenitors responsible for the developing pancreas. They give rise to both the endocrine and exocrine cells. Exocrine cells constitute the acinar cells and the ductal cells. The endocrine cells constitute the beta cells which make insulin, alpha cells which secrete glucagon, delta cells which secrete somatostatin and the PP-cells which secrete pancreatic polypeptide. In embodiments, progenitor cells used herein refer to endocrine precursors. The endocrine precursors are a committed group of progenitors that develop into all of the endocrine cells in the pancreas.

The term “fully differentiated cells” refers to cells that have completed the differentiation process and have become specialized in order to perform a specific function. In embodiments, fully differentiated alpha cells refer to alpha cells that are specialized to secret glucagon. In embodiments, fully differentiated beta cells refer to beta cells that are specialized to secret insulin. In embodiments, fully differentiated delta cells refer to delta cells that are specialized to secret somatostatin.

In the context of experimentation or measurements, the term “ex vivo” refers to experimentation or measurements done in or on tissue from an organism in an external environment. In embodiments, there is minimal alteration of natural conditions. Ex vivo conditions allow experimentation on an organism's cells or tissues under more controlled conditions than is possible in in vivo experiments (in the intact organism), at the expense of altering the “natural” environment. In embodiments, the term ex vivo means that the samples to be tested have been extracted from the organism. In embodiments, an ex vivo culture used herein refers to a culture that involve living cells or tissues taken from an organism. In embodiments, the living cells or tissue are cultured in a sterile vessel (such as a flask, plate, or bottle). In embodiments, the living cells or tissue are cultured in a laboratory apparatus, usually under sterile conditions with no alterations. In embodiments, the living cells or tissue are cultured for up to 24 hours to obtain sufficient cells for an experiments, assay, or for treatment.

The oxygen consumption rate (OCR) of cells is an important indicator of normal cellular function. It is used as a parameter to study mitochondrial function as well as a marker of factors triggering the switch from healthy oxidative phosphorylation to aerobic glycolysis in cancer cells. In embodiments, OCR is an indicator or predictor of islets transplantation success. OCR level can be determined according to any methods available in the art. In embodiments, OCR level can be determined according to the method described in the examples presented herein.

### Exemplary Cultures, Compositions, and Kits

In an aspect, included herein is an isolated islet cell or an isolated islet cell population that has been cultured ex vivo according to a method provided herein.

In an aspect, provided herein is an ex vivo culture. In embodiments, the ex vivo culture includes an islet cell population in a culture medium, wherein the culture medium comprises EGF or a fragment thereof. In embodiments, the islet cell population is a fully differentiated islet cell population.

In an aspect, the disclosures herewith provide an ex vivo culture having a plurality or population of fully differentiated islet cells in a culture medium, where the culture medium can contain EGF or a fragment thereof.

In an aspect, provided herein is an ex vivo culture having a plurality or population of islet cells in a culture medium, where the culture medium can contain EGF or a fragment thereof.

In an aspect, included herein is a culture medium for increasing the viability or of an islet cell population, and/or the suitability thereof for administration to a subject with a disease associated with loss of islet cell function or viability. In embodiments, the culture medium includes EGF or a fragment thereof.

In an aspect, included herein is a kit for increasing the viability or of an islet cell population, and/or the suitability thereof for administration to a subject with a disease associated with loss of islet cell function or viability. In embodiments, the kit includes a vessel or container containing culture medium and another vessel or container EGF or a fragment thereof.

In embodiments, the population of islet cells is obtained from one or more donor subjects. In embodiments, the population includes fully differentiated islet cells. In embodiments, the islet cell population has been obtained from one, two, or three isolated islets. In embodiments, the one or more islets has been isolated from one, two, or three donor subjects. In embodiments, the islet cell population has been obtained from one or more isolated islets. In embodiments, the one or more islets have been isolated from one or more donor subjects. In embodiments, the islet cell population has been obtained from one isolated islet. In embodiments, the one or more islets have been isolated from one donor subject. In embodiments, the donor subject is human. In embodiments, the donor subject is deceased.

In embodiments, the culture medium is a liquid culture medium.

In embodiments, the culture medium comprises gastrin. In embodiments, the culture medium does not comprise gastrin. In embodiments, the gastrin is recombinant gastrin.

In embodiments, the culture medium comprises glucagon-like peptide-1 (GLP-1). In embodiments, the culture medium does not comprise GLP-1. In embodiments, the GLP-1 is recombinant GLP-1.

In embodiments, the culture medium comprises a GLP-1 receptor agonist. In embodiments, culture medium does not comprise a GLP-1 receptor agonist. In embodiments, the GLP-1 receptor agonist is exenatide, liraglutide, lixisenatide, albiglutide, dulaglutide, semaglutide, or taspoglutide.

In embodiments, culture medium is any medium disclosed herein. In embodiments, the culture medium is any medium known in the art for culturing islet cells. In embodiments, the culture medium is used to maintain the islet cell population for a period of time.

In embodiments, the culture medium includes: glutathione, vitamin E or equivalent, such as Tocopherols, L-glutamine, zinc chloride, and one or more ligands of the epidermal growth factor receptor (EGFR), including natural or recombinant transforming growth factor-alpha or other ligand or fragments of them.

In embodiments, the culture medium used in the method described herein comprises, consists essentially of, or consists of EGF or a fragment thereof and CMRL 1066, Supplemented, CIT Modification (Corning®, 98-304 CV), and human serum albumin.

In embodiments, the culture medium includes: glutathione, vitamin E or equivalent, such as Tocopherols, L-glutamine, zinc chloride, and one or more ligands of the epidermal growth factor receptor (EGFR), including natural or recombinant transforming growth factor-alpha or other ligand or fragments of them.

In embodiments, the culture medium used in the method described herein comprises, consists essentially of, or consists of EGF or a fragment thereof and CMRL 1066, Supplemented, CIT Modification (Corning®, 98-304 CV), and human serum albumin.

In embodiments, the culture medium includes:

| | | Amount/ | | Amount/ | | Final | |
|---|---|---|---|---|---|---|---|
| Materials | SOP#/Source | 500 mL | | 1 L | | Concentration | |
| CMRL 1066 | Mediatech, 99-663-CV | 500 | mL | 2 × 500 | | N/A | |
| | | | | mL bottle | | | |
| 1M HEPES | Mediatech, 25-060-Cl | 14.5 | mL | 29 | mL | 25 | mM |
| 25% Human Serum Albumin, HSA | QCS021B | 26 | mL | 52 | mL | 1.125% | |
| ITS stock solution (stable at −20° C. | MFR068C | 0.8 | mL | 1.6 | mL | 6.25 | μg/mL |
| for 3 months after reconstitution) | | | | | | | |
| 200 mM L-Alanyl-L-Glutamine | Mediatech, 25-015-Cl | 6 | mL | 12 | mL | 2 | mM |
| 60 μg/ml Linoleic acid solution | MFR069C | 52 | μl | 103 | μl | 5.35 | μg/L |
| 1M NaOH | QCS033B | 0.5-1.5 | mL | 1-3 | mL | Adjust pH | |
| | | | | | | to 7.2-7.4 | |
| 2.5M Nicotinamide solution | MFR061C | 2.3 | mL | 4.6 | mL | 10 | mM |
| 100 mM Sodium Pyruvate | Mediatech, 25-000-Cl | 29 | mL | 58 | mL | 5 | mM |
| 50 mM Trolox solution | MFR060D | 115 | μl | 230 | μl | 10 | μM |
| 0.2M Zinc Sulfate | MFR070D | 48 | μl | 96 | μl | 16.7 | μM |

In embodiments, CMRL 1066 Formulation is (concentrations are listed in mg/l):

Components

Inorganic Salts

| | |
|---|---|
| CaCl2 (anhydrous) | 200 |
| KCl | 400 |
| MgSO4 (anhydrous) | 97.70 |
| NaCl | 6800 |
| NaH2PO4•H₂O | 140 |
| NaHCO₃ | 2200 |

Amino Acids

| | |
|---|---|
| L-Alanine | 25 |
| L-Arginine•HCL | 70 |
| L-Aspartic Acid | 30 |
| L-Cysteine•HCl•H₂O | 260 |
| L-Cystine•2HCl | 26 |
| L-Glutamic Acid | 75 |
| Glycine | 50 |
| L-Histidine•HCl•H₂O | 20 |
| Hydroxy-L-Proline | 10 |
| L-Isoleucine | 20 |
| L-Leucine | 60 |
| L-Lysine•HCl | 70 |
| L-Methionine | 15 |
| L-Phenylalanine | 25 |
| L-Proline | 40 |
| L-Serine | 25 |
| L-Threonine | 30 |
| L-Tryptophan | 10 |
| L-Tyrosine•2Na•2H₂O | 58 |
| L-Valine | 25 |

Vitamins

| | |
|---|---|
| Biotin | 0.01 |
| Folic Acid | 0.01 |
| Riboflavin | 0.01 |
| Ascorbic Acid | 50.00 |
| D-Ca-Pantothenate | 0.01 |
| Choline Chloride | 0.50 |
| i-Inositol | 0.05 |
| Nicotinic Acid | 0.025 |
| Nicotinamide | 0.025 |
| PABA | 0.05 |
| Pyridoxine•HCl | 0.05 |
| Thiamine•HCl | 0.01 |
| Thiamine pyrophosphate, Na | 1.00 |

Other

| | |
|---|---|
| Thymidine | 10.00 |
| 2′-Deoxyadenosine•H₂O | 10.00 |
| 2′-Deoxycytidine•HCl | 10.00 |
| 2′-Deoxyguanosine•H₂O | 10.00 |
| 5-Methyl-2′-Deoxycytidine | 0.10 |
| Uridine-5′-triphosphate•3Na•hydrate | 1.00 |
| Cholesterol | 0.20 |
| Polysorbate 80 | 5.00 |
| Coenzyme A Li₃ Salt•2H₂O | 2.50 |
| b-NAD•hydrate | 7.00 |
| b-NADP•Na•4H₂O | 1.00 |
| FAD Disodium Salt | 1.00 |
| Dextrose | 1000 |
| Glutathione (reduced) | 10.00 |
| Sodium acetate | 50.00 |
| Sodium glucuronate•H₂O | 4.20 |

Add

| | |
|---|---|
| L-Glutamine Powder (mg/L) | 100.00 |
| 200 mM Solution (mL/L) | 3.42 |

In embodiments, the culture medium comprises serum. In embodiments, the cell culture medium does not comprise serum.

In embodiments, the islet cell population comprises, consists of, or consists essentially of beta cells, alpha cells, delta cells, progenitor cells, or any mixture thereof. In embodiments, the islet cell population comprises, consists of, or consists essentially of beta cells. In embodiments, the islet cell population comprises, consists of, or consists essentially of alpha cells. In embodiments, the islet cell population comprises, consists of, or consists essentially of delta cells. In embodiments, the islet cell population comprises, consists of, or consists essentially of progenitor cells. In embodiments, the beta cells, alpha cells, and/or delta cells are fully differentiated.

In embodiments, the fully differentiated islet cells contained in a ex vivo culture according to the present disclosure are fully differentiated beta cells, fully differentiated alpha, fully differentiated delta cells, or a mixture thereof.

In embodiments, the culture medium comprises from 0.001 to 10 ng/ml, from 0.001 to 0.01 ng/ml, from 0.001 to 0.1 ng/ml, from 0.001 to 1 ng/ml, from 0.01 to 1 ng/ml, from 0.1 to 1 ng/ml, from 0.1 to 0.5 ng/ml, from 0.001 to 0.5 ng/ml, from 0.5 to 15 ng/ml, from 1 to 10 ng/ml, from 2 to 10 ng/ml, from 3 to 10 ng/ml, from 4 to 10 ng/ml, from 5 to 10 ng/ml, from 6 to 10 ng/ml, from 7 to 10 ng/ml, from 8 to 10 ng/ml, from 9 to 10 ng/ml, from 1 to 9 ng/ml, from 1 to 8 ng/ml, from 1 to 7 ng/ml, from 1 to 6 ng/ml, from 1 to 5 ng/ml, from 1 to 4 ng/ml, from 1 to 3 ng/ml, from 1 to 2 ng/ml, from 1 to 5 ng/ml, from 2 to 5 ng/ml, or from 10 to 15 ng/ml EGF or a fragment thereof. In embodiments, the islet tissue or islet cell population is maintained, cultured, or stored in a medium comprising about 0.001, 0.01, 0.1, 0.5, 1, 2, 3, 4, 5, 6, 7, 8, 9, 10, 11, 12, 13, 14, or 15 ng/ml EGF or a fragment thereof. In embodiments, the islet tissue or islet cells are maintained, cultured, or stored in a medium comprising from 2 to 10 ng/ml EGF or a fragment thereof.

In embodiments, the EGF or fragment thereof is present in a culture medium at an amount of about 2 to 10 ng/ml. In embodiments, the EGF or fragment thereof can be present in a culture medium at a concentration of 2 to 10 ng/ml. In embodiments, the EGF or fragment thereof is present in a culture medium at an amount of about 0.1 ng/ml, about 0.5 ng/ml, about 1 ng/ml, about 1.5 ng/ml, about 2.0 ng/ml, about 2.5 ng/ml, about 3.0 ng/ml, about 3.5 ng/ml, about 4.0 ng/ml, about 4.5 ng/ml, about 5.0 ng/ml, about 5.5 ng/ml, about 6.0 ng/ml, about 6.5 ng/ml, about 7.0 ng/ml, about 7.5 ng/ml, about 8.0 ng/ml, about 8.5 ng/ml, about 9.0 ng/ml, about 9.5 ng/ml, about 10.0 ng/ml, about 10.5 ng/ml, about 11.0 ng/ml, about 11.5 ng/ml, about 12.0 ng/ml, about 12.5 ng/ml, about 13.0 ng/ml, about 13.5 ng/ml, about 14.0 ng/ml, about 14.5 ng/ml, about 15.0 ng/ml, or more than about 15.0 ng/ml, or any intervening values of the foregoing-listed amounts.

In embodiments, the EGF or fragment thereof can be present in a culture medium at a concentration of about 0.1-200 ng/ml (e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/ml).

In embodiments, the EGF or fragment thereof is human EGF or a fragment thereof. In embodiments, the human EGF has the amino acid sequence of SEQ ID NO: 1. In embodiments, the EGF has an amino acid sequence that is about 90%, about 91%, about 92%, about 93%, about 94%, about 95%, about 96%, about 97%, about 98%, about 99% identity to SEQ ID NO: 1. In embodiments, the EGF has 5, 4, 3, 2, 1, or 0 deletions compared to SEQ ID NO: 1. In embodiments, the EGF has 5, 4, 3, 2, 1, or 0 insertions compared to SEQ ID NO: 1. In embodiments, the EGF has 5, 4, 3, 2, 1, or 0 substitutions compared to SEQ ID NO: 1. In embodiments, all of the substitutions are conservative substitutions. In embodiments, the EGF fragment has the amino acid sequence of SEQ ID NO: 2 or SEQ ID NO: 3. In some embodiments, the fragment of EGF has an amino acid sequence that is about 90%, about 91%, about 92%, about 93%, about 94%, about 95%, about 96%, about 97%, about 98%, about 99% identity to SEQ ID NO: 2 or SEQ ID NO: 3. In embodiments, the EGF fragment has 5, 4, 3, 2, 1, or 0 deletions compared to SEQ ID NO: 2 or 3. In embodiments, the EGF fragment has 5, 4, 3, 2, 1, or 0 insertions compared to SEQ ID NO: 2 or 3. In embodiments, the EGF fragment has 5, 4, 3, 2, 1, or 0 substitutions compared to SEQ ID NO: 2 or 3. In embodiments, all of the substitutions are conservative substitutions.

### Exemplary Culture, Preparation, and Treatment Methods

In an aspect, provided herein is a method of culturing a population of islet cells. In embodiments, the method includes culturing the population of islet cells in a culture medium having epidermal growth factor (EGF) or a fragment thereof.

In an aspect, provided herein is a method of producing a cultured islet cell population. In embodiments, the method includes culturing an islet cell population in a culture medium that includes EGF or a fragment thereof, thereby forming the cultured islet cell population.

In an aspect, provided herein is a method of increasing the viability or function of an islet cell population. In embodiments, the method includes contacting the islet cell population with an effective amount of EGF or a fragment thereof.

In an aspect, a method of increasing the survival or recovery of islet cells isolated from islet tissue is provided. In embodiments, the method comprises contacting the islet cells with an effective amount of EGF or a fragment thereof.

In an aspect, a method of increasing the recovery of viable islet cells from a subject. In embodiments, the method includes contacting the islet cells with EGF or a fragment thereof.

In an aspect, included herein is a method of administering (e.g., transplanting) to a subject in need thereof an islet cell population. In embodiments, the islet cell population has been cultured ex vivo according to a method provided herein.

In embodiments, the islet cell population forms part of islet tissue. In embodiments, the islet cell population has been isolated from islet tissue.

In an aspect, included herein is a method of isolating an islet cell population from islet tissue. In embodiments, the method comprises contacting the islet tissue (e.g., by incubation and/or perfusion) with at least one enzyme that digests the tissue or a portion thereof (such as one or more components of a basement membrane in the tissue). In embodiments, the enzyme is a collagenase. In embodiments, the method comprises mechanically separating cells in the tissue from each other. In embodiments, the tissue is contacted with EGF (e.g., by incubation and/or perfusion) or a fragment thereof before, during, and/or after it is contacted with the at least one enzyme. In embodiments, the tissue is contacted with EGF (e.g., by incubation and/or perfusion) or a fragment thereof before, during, and/or after the cells are mechanically separated. In embodiments, the islet cell population is isolated from the tissue after the tissue is contacted with the enzyme and/or the cells are mechanically separated from each other. In embodiments, the islet cell population is contacted with EGF after it is isolated from the islet tissue. In embodiments, at least 75%, 80%, 85%, 90%, or 95% of the islet cells in the islet tissue are isolated from the islet tissue and are viable. In embodiments, 75-85%, 85-95%, 75-95%, 75-100%, 85-100%, or 95-100% of the islet cells in the islet tissue are isolated from the islet tissue and are viable. In embodiments, at least 75%, 80%, 85%, 90%, or 95% of the islet cells in the islet tissue are isolated from the islet tissue and are suitable for transplantation. In embodiments, 75-85%, 85-95%, 75-95%, 75-100%, 85-100%, or 95-100% of the islet cells in the islet tissue are isolated from the islet tissue and are suitable for transplantation.

In embodiments, the islet cell population comprises, consists of, or consists essentially of beta cells, alpha cells, delta cells, progenitor cells, or any mixture thereof. In embodiments, the islet cell population comprises, consists of, or consists essentially of beta cells. In embodiments, the islet cell population comprises, consists of, or consists essentially of alpha cells. In embodiments, the islet cell population comprises, consists of, or consists essentially of delta cells. In embodiments, the islet cell population comprises, consists of, or consists essentially of progenitor cells. In embodiments, the beta cells, alpha cells, and/or delta cells are fully differentiated.

In embodiments, the culture medium used in the methods described herein can be an ex vivo culture medium.

In embodiments, the culture medium used in the methods described herein can be a liquid culture medium.

In embodiments, an islet cell population is cultured under normoglycemic conditions. In embodiments, maintaining, culturing, or storing islet cells to be transplanted in a culture or storage medium with a normal glycemic concentration avoids or minimizes potential modifications to the cells. In embodiments, methods provided herein do not include culturing islet cells in hyperglycemic conditions, which may modify the islets making them sensitive to the culturing, which may have to be used chronically. In embodiments, the medium comprises from 3.9 to 5.5 mM sugar. In embodiments, the medium comprises about 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5 mM sugar. In embodiments, the medium comprises about 5.5 mM sugar. In embodiments, the medium comprises from 3.9 to 5.5 mM glucose. In embodiments, the medium comprises about 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5 mM glucose. In embodiments, the medium comprises about 5.5 mM glucose. In embodiments, islet tissue or cells are cultured or stored (e.g., for shipment or prior to transplant) in well-defined media and defined key components (such as CMRL Supplemented, CIT Modification, and Human Serum Albumin) to which EGF or a fragment thereof is added.

Typically, islet cells have not been used from donors with a HbA1c greater than 5.6%. In embodiments, methods and compositions provided herein extend the range of potential donors from normal donors to donors with a HbA1c greater than 5.6%. In embodiments, methods and compositions provided herein extend the range of potential donors from donors with a HbA 1 c equal to or less than 5.6%, to donors with a HbA1c greater than 5.6%. In embodiments, methods and compositions provided herein are used to culture islet cells from donors with a HbA1c equal to or less than 5.6%. In embodiments, methods and compositions provided herein are used to culture islet cells from donors with a HbA1c greater than 5.6%.

In embodiments, maintaining, culturing, or storing islet tissue or an islet cell population in a composition comprising EGF or a fragment thereof (such as in a culture medium) for a relatively short period of time improves the viability or function of the islet tissue or islet cell population after transplantation. In embodiments, these benefits persist long after transplantation, e.g., at least about 1, 2, 3, or 4 weeks, at least about 1, 2, 3, 4, 5, 6, 7, 8, 9, 10, 11, or 12 months, at least about 1, 2, 3, 4, or 5 years, from about 1 to 4 weeks, from about 1 to 12 months, or from about 1 to 5 years. In embodiments, the islet tissue or islet cell population is contacted with EGF or a fragment thereof (e.g., in a culture medium) for less than about 96, less than about 72, less than about 48, less than about 36, or less than about 24 hours. In embodiments, cells from an islet cell population are administered to a subject in need thereof within about 96, 72, 48, 36, or 24 hours after the islet cell population is first contacted with recombinant EGF or a fragment thereof in a culture medium. In embodiments, islet tissue is perfused with, or maintained or cultured or stored within, a medium comprising recombinant EGF or a fragment thereof before islet cells are obtained (e.g., isolated) from the tissue. In embodiments, an islet cell population is obtained (e.g., isolated) from islet tissue and then cultured, maintained or stored within a medium comprising EGF or a fragment thereof. In embodiments, islet tissue is stored or shipped in medium comprising EGF or a fragment thereof for less than about 96, 72, 48, 36, or 24 hours before an islet cell population is obtained from the islet tissue. In embodiments, islet cells are stored or shipped in medium comprising EGF or a fragment thereof for less than about 96, 72, 48, 36, or 24 hours before being administered to a subject. In embodiments, the total amount of time that islet tissue or islet cells are in a composition comprising EGF or a fragment thereof prior to being administered is less than about 96, 72, 48, 36, or 24 hours. In embodiments, an islet cell population is treated for less than about 96, 72, 48, 36, or 24 hours after processing (e.g., isolation from islet tissue). In embodiments, such short treatment periods result in cells with increased viability and/or function while reducing or preventing culture contamination (due to reducing the time that a culture may inadvertently become contaminated). In embodiments, such short treatment periods reduce the amount of time for cells to become modified due to ex vivo growth conditions. In embodiments, such short treatment periods reduce the amount of time for cells to lose viability or die during ex vivo culture.

In embodiments, the EGF or fragment thereof is recombinant EGF or a recombinant EFG fragment. In embodiments, the EGF has the same amino acid sequence as the EGF that naturally occurs in the donor and subject (e.g., human islet cells are contacted with human EGF). In embodiments, the EGF fragment has an amino acid sequence that is part of the amino acid sequendce of EGF that naturally occurs in the donor and subject. In embodiments, an islet cell population is maintained, cultured, or stored in a medium comprising an amount of EGF or a fragment thereof that reduces the immunogenicity of the islet cells. In embodiments, an islet cell population is maintained, cultured, or stored in a medium comprising an amount of EGF or a fragment thereof that reduces the expression of a human leukocyte antigen (HLA) class I antigen on the surface of the islet cells. In embodiments, an islet cell population is maintained, cultured, or stored in a medium comprising an amount of EGF or a fragment thereof that reduces the expression of HLA-A, HLA-B, and/or HLA-C on the surface of cells in the population. In embodiments, the islet tissue or islet cell population is maintained, cultured, or stored in a medium comprising from 0.001 to 10 ng/ml, from 0.001 to 0.01 ng/ml, from 0.001 to 0.1 ng/ml, from 0.001 to 1 ng/ml, from 0.01 to 1 ng/ml, from 0.1 to 1 ng/ml, from 0.1 to 0.5 ng/ml, from 0.001 to 0.5 ng/ml, from 0.5 to 15 ng/ml, from 1 to 10 ng/ml, from 2 to 10 ng/ml, from 3 to 10 ng/ml, from 4 to 10 ng/ml, from 5 to 10 ng/ml, from 6 to 10 ng/ml, from 7 to 10 ng/ml, from 8 to 10 ng/ml, from 9 to 10 ng/ml, from 1 to 9 ng/ml, from 1 to 8 ng/ml, from 1 to 7 ng/ml, from 1 to 6 ng/ml, from 1 to 5 ng/ml, from 1 to 4 ng/ml, from 1 to 3 ng/ml, from 1 to 2 ng/ml, from 1 to 5 ng/ml, from 2 to 5 ng/ml, or from 10 to 15 ng/ml EGF or a fragment thereof. In embodiments, the islet tissue or islet cell population is maintained, cultured, or stored in a medium comprising about 0.001, 0.01, 0.1, 0.5, 1, 2, 3, 4, 5, 6, 7, 8, 9, 10, 11, 12, 13, 14, or 15 ng/ml EGF or a fragment thereof. In embodiments, the islet tissue or islet cells are maintained, cultured, or stored in a medium comprising from 2 to 10 ng/ml EGF or a fragment thereof.

In embodiments, the EGF or fragment thereof used in the methods described herein is present in a culture medium at an amount of about 2 to 10 ng/ml. In embodiments, the EGF or fragment thereof used in the methods described herein can be present in a culture medium at a concentration of 2 to 10 ng/ml. In embodiments, the EGF or fragment thereof used in the methods described herein can be present in a culture medium at an amount of about 0.1 ng/ml, about 0.5 ng/ml, about 1 ng/ml, about 1.5 ng/ml, about 2.0 ng/ml, about 2.5 ng/ml, about 3.0 ng/ml, about 3.5 ng/ml, about 4.0 ng/ml, about 4.5 ng/ml, about 5.0 ng/ml, about 5.5 ng/ml, about 6.0 ng/ml, about 6.5 ng/ml, about 7.0 ng/ml, about 7.5 ng/ml, about 8.0 ng/ml, about 8.5 ng/ml, about 9.0 ng/ml, about 9.5 ng/ml, about 10.0 ng/ml, about 10.5 ng/ml, about 11.0 ng/ml, about 11.5 ng/ml, about 12.0 ng/ml, about 12.5 ng/ml, about 13.0 ng/ml, about 13.5 ng/ml, about 14.0 ng/ml, about 14.5 ng/ml, about 15.0 ng/ml, or more than about 15.0 ng/ml, or any intervening values of the foregoing-listed amounts.

In embodiments, the EGF or fragment thereof used in the methods and compositions described herein can be present in a culture medium at a concentration of about 0.1-200 ng/ml (e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/ml).

In embodiments, the islet tissue or population of islet cells is contacted with gastrin. In embodiments, the islet tissue or population of islet cells is not contacted with gastrin. In embodiments, the gastrin is recombinant gastrin.

In embodiments, the islet tissue or population of islet cells is contacted with glucagon-like peptide-1 (GLP-1). In embodiments, the islet tissue or population of islet cells is not contacted with GLP-1. In embodiments, the GLP-1 is recombinant GLP-1. In embodiments, the GLP-1 is endogenous GLP-1.

In embodiments, the islet tissue or population of islet cells is contacted with a GLP-1 receptor agonist. In embodiments, the islet tissue or population of islet cells is not contacted with a GLP-1 receptor agonist. In embodiments, the GLP-1 receptor agonist is exenatide, liraglutide, lixisenatide, albiglutide, dulaglutide, semaglutide, or taspoglutide.

In embodiments, culture medium is any medium disclosed herein. In embodiments, the culture medium is any medium known in the art for culturing islet cells, plus EGF or a fragment thereof. In embodiments, the culture medium is used to maintain the islet cell population for a period of time. In embodiments, the culture medium is used to ship the islet cell population from one location (e.g., the location of the donor) to another (e.g., the location of the subject to be treated).

In embodiments, the culture medium includes: glutathione, vitamin E or equivalent, such as Tocopherols, L-glutamine, zinc chloride, and one or more ligands of the epidermal growth factor receptor (EGFR), including natural or recombinant transforming growth factor-alpha or other ligand or fragments of them.

In embodiments, the culture medium used in the method described herein comprises, consists essentially of, or consists of EGF or a fragment thereof and CMRL 1066, Supplemented, CIT Modification (Corning®, 98-304 CV), and human serum albumin.

In embodiments, the culture medium used in the methods described herein includes:

| | | Amount/ | | Amount/ | | Final | |
|---|---|---|---|---|---|---|---|
| Materials | SOP#/Source | 500 mL | | 1 L | | Concentration | |
| CMRL 1066 | Mediatech, 99-663-CV | 500 | mL | 2 × 500 | | N/A | |
| | | | | mL bottle | | | |
| 1M HEPES | Mediatech, 25-060-Cl | 14.5 | mL | 29 | mL | 25 | mM |
| 25% Human Serum Albumin, HSA | QCS021B | 26 | mL | 52 | mL | 1.125% | |
| ITS stock solution (stable at −20° C. | MFR068C | 0.8 | mL | 1.6 | mL | 6.25 | μg/mL |
| for 3 months after reconstitution) | | | | | | | |
| 200 mM L-Alanyl-L-Glutamine | Mediatech, 25-015-Cl | 6 | mL | 12 | mL | 2 | mM |
| 60 μg/ml Linoleic acid solution | MFR069C | 52 | μl | 103 | μl | 5.35 | μg/L |
| 1M NaOH | QCS033B | 0.5-1.5 | mL | 1-3 | mL | Adjust pH | |
| | | | | | | to 7.2-7.4 | |
| 2.5M Nicotinamide solution | MFR061C | 2.3 | mL | 4.6 | mL | 10 | mM |
| 100 mM Sodium Pyruvate | Mediatech, 25-000-Cl | 29 | mL | 58 | mL | 5 | mM |
| 50 mM Trolox solution | MFR060D | 115 | μl | 230 | μl | 10 | μM |
| 0.2M Zinc Sulfate | MFR070D | 48 | μl | 96 | μl | 16.7 | μM |

In embodiments, CMRL 1066 Formulation is (concentrations are listed in mg/l):

Components

Inorganic Salts

| | |
|---|---|
| CaCl2 (anhydrous) | 200 |
| KCl | 400 |
| MgSO4 (anhydrous) | 97.70 |
| NaCl | 6800 |
| NaH2PO4•H₂O | 140 |
| NaHCO₃ | 2200 |

Amino Acids

| | |
|---|---|
| L-Alanine | 25 |
| L-Arginine•HCL | 70 |
| L-Aspartic Acid | 30 |
| L-Cysteine•HCl•H₂O | 260 |
| L-Cystine•2HCl | 26 |
| L-Glutamic Acid | 75 |
| Glycine | 50 |
| L-Histidine•HCl•H₂O | 20 |
| Hydroxy-L-Proline | 10 |
| L-Isoleucine | 20 |
| L-Leucine | 60 |
| L-Lysine•HCl | 70 |
| L-Methionine | 15 |
| L-Phenylalanine | 25 |
| L-Proline | 40 |
| L-Serine | 25 |
| L-Threonine | 30 |
| L-Tryptophan | 10 |
| L-Tyrosine•2Na•2H₂O | 58 |
| L-Valine | 25 |

Vitamins

| | |
|---|---|
| Biotin | 0.01 |
| Folic Acid | 0.01 |
| Riboflavin | 0.01 |
| Ascorbic Acid | 50.00 |
| D-Ca-Pantothenate | 0.01 |
| Choline Chloride | 0.50 |
| i-Inositol | 0.05 |
| Nicotinic Acid | 0.025 |
| Nicotinamide | 0.025 |
| PABA | 0.05 |
| Pyridoxine•HCl | 0.05 |
| Thiamine•HCl | 0.01 |
| Thiamine pyrophosphate, Na | 1.00 |

Other

| | |
|---|---|
| Thymidine | 10.00 |
| 2′-Deoxyadenosine•H₂O | 10.00 |
| 2′-Deoxycytidine•HCl | 10.00 |
| 2′-Deoxyguanosine•H₂O | 10.00 |
| 5-Methyl-2′-Deoxycytidine | 0.10 |
| Uridine-5′-triphosphate•3Na•hydrate | 1.00 |
| Cholesterol | 0.20 |
| Polysorbate 80 | 5.00 |
| Coenzyme A Li₃ Salt•2H₂O | 2.50 |
| b-NAD•hydrate | 7.00 |
| b-NADP•Na•4H₂O | 1.00 |
| FAD Disodium Salt | 1.00 |
| Dextrose | 1000 |
| Glutathione (reduced) | 10.00 |
| Sodium acetate | 50.00 |
| Sodium glucuronate•H₂O | 4.20 |

Add

| | |
|---|---|
| L-Glutamine Powder (mg/L) | 100.00 |
| 200 mM Solution (mL/L) | 3.42 |

In embodiments, the islet cell population to be cultured by the methods described herein is obtained from one, two, or three isolated islets. In embodiments, the one or more islets are isolated from one, two, or three donor subjects. In embodiments, the islet cell population to be cultured by the methods described herein is obtained from one or more isolated islets. In embodiments, the one or more islets are isolated from one or more donor subjects. In embodiments, the islet cell population to be cultured by the methods described herein is obtained from one isolated islet. In embodiments, the one or more islets are isolated from one donor subject. In embodiments, the donor subject is human. In embodiments, the donor subject is deceased.

In embodiments, transplantation is only possible if the islet equivalent (IEQ) is >5000 IEQ/kg of the recipient's body weight. Between 300000 to 400000 islet equivalent (IEQ) can be obtained from a normal donor, if a pancreas is maintained under optimal conditions. However, during the required in vitro processing of islets before transplantation, there is up to 30% loss of cells, due to the high sensitivity of islets to the stress conditions. In embodiments, the exogenous administration of EGF may improves the recovery of IEQ. In embodiments, less than 30% of the islet cells are lost during processing. In embodiments, less than 25% of the islet cells are lost during processing. In embodiments, less than 20% of the islet cells are lost during processing. In embodiments, less than 15% of the islet cells are lost during processing. In embodiments, less than 10% of the islet cells are lost during processing. In embodiments, less than 5% of the islet cells are lost during processing.

In embodiments, the islet cell population be cultured by the methods described herein can be fully differentiated islet cells or a plurality of progenitor cells.

In embodiments, the islet cell population to be cultured by the methods described herein can be a plurality of fully differentiated beta cells, a plurality of fully differentiated alpha cells, a plurality of fully differentiated delta cells, a plurality of progenitor cells, or a mixture thereof. In embodiments, the plurality of islet cells can be a plurality of human islet cells.

In embodiments, the EGF or fragment thereof used in the methods described herein is human EGF or a fragment thereof. In embodiments, the human EGF has the amino acid sequence of SEQ ID NO: 1. In embodiments, the EGF has an amino acid sequence that is about 90%, about 91%, about 92%, about 93%, about 94%, about 95%, about 96%, about 97%, about 98%, about 99% identity to SEQ ID NO: 1. In embodiments, the EGF has 5, 4, 3, 2, 1, or 0 deletions compared to SEQ ID NO: 1. In embodiments, the EGF has 5, 4, 3, 2, 1, or 0 insertions compared to SEQ ID NO: 1. In embodiments, the EGF has 5, 4, 3, 2, 1, or 0 substitutions compared to SEQ ID NO: 1. In embodiments, all of the substitutions are conservative substitutions. In embodiments, the EGF fragment has the amino acid sequence of SEQ ID NO: 2 or SEQ ID NO: 3. In some embodiments, the fragment of EGF has an amino acid sequence that is about 90%, about 91%, about 92%, about 93%, about 94%, about 95%, about 96%, about 97%, about 98%, about 99% identity to SEQ ID NO: 2 or SEQ ID NO: 3. In embodiments, the EGF fragment has 5, 4, 3, 2, 1, or 0 deletions compared to SEQ ID NO: 2 or 3. In embodiments, the EGF fragment has 5, 4, 3, 2, 1, or 0 insertions compared to SEQ ID NO: 2 or 3. In embodiments, the EGF fragment has 5, 4, 3, 2, 1, or 0 substitutions compared to SEQ ID NO: 2 or 3. In embodiments, all of the substitutions are conservative substitutions.

In embodiments, the EGF or fragment thereof used in the methods described herein increases oxygen consumption rate (OCR) of the cultured islet tissue or islet cell population according to the methods described herein compared to the islet tissue or islet cell population that is originally obtained. In embodiments, the EGF or fragment thereof increases OCR at about 0.5, 0.6, 0.7, 0.8, 0.9, 1, 2, 3, 4, 5, 6, 7, 8, 9, or 10-fold when compared to the OCR of the islet tissue or islet cell population before culturing.

In embodiments, the EGF or fragment thereof is present in the culture medium at an amount sufficient to increase oxygen consumption rate of the cultured islet tissue or islet cell population. In embodiments, the EGF or a fragment thereof is present in the culture medium at an amount sufficient to increase oxygen consumption rate of the cultured islets or islet cells for about at least 0.5, 0.6, 0.7, 0.8, 0.9, 1, 2, 3, 4, 5, 6, 7, 8, 9, or 10-fold when compared to the OCR of the islets or islet cells before culturing.

In embodiments, the cultured islet tissue or islet cell population according to the methods described herein can be more bioactive or has a higher function (e.g., about 0.5, 0.6, 0.7, 0.8, 0.9, 1, 2, 3, 4, 5, 6, 7, 8, 9, or 10-fold higher) relative to the plurality of islets or islet cells. In embodiments, the bioactive or function of islets or islet cells is determined via the OCR level: the higher OCR level indicates higher bioactive or function of islets or islet cells. In embodiments, the bioactivity or function of the islet tissue or islet cell population is determined via the level of the peptide hormone (e.g., glucagon by alpha cells, insulin by beta cells and somatostatin or Ucn3 by delta cells) released from the islets or islet cells.

In an aspect, a method of increasing the viability or function of an islet cell population is provided, the method comprising culturing the islet cell population in a culture medium comprising an effective amount of EGF or a fragment thereof.

In an aspect, a method of increasing the viability or function of an islet cell population is provided, the method comprising culturing the islet cell population in a culture medium comprising an effective amount of EGF or a fragment thereof for up to 72 hours.

In an aspect, a method of increasing the viability or function of an islet cell population is provided, the method comprising culturing the islet cell population in a culture medium comprising an effective amount of EGF or a fragment thereof, wherein the effective amount is 2-10 ng/ml.

In an aspect, a method of increasing the viability or function of an islet cell population is provided, the method comprising culturing the islet cell population in a culture medium comprising an effective amount of EGF or a fragment thereof, wherein the culture medium comprises glucose in a concentration of from 3.9 to 5.5 mM.

In an aspect, a method of increasing the viability or function of an islet cell population is provided, the method comprising culturing the islet cell population in a culture medium comprising an effective amount of epidermal growth factor (EGF) or a fragment thereof, wherein the islet cell population has been isolated from a subject with a Hemoglobin A1c (HbA1c) greater than 5.6%

In aspects, an EGF variant is used rather than EGF. In embodiments, the EGF variant is a fusion protein that includes EGF or a variant thereof covelantly bound to gastrin, GLP-1, or a GLP-1 receptor agonist.

In an aspect, provided herein is a method of treating a disease in a subject in need thereof. In embodiments, the method includes administering to the subject an effective amount of islet tissue or an islet cell population. In embodiments, the method includes administering an effective amount of EGF or a fragment thereof to the subject. In embodiments, the method includes administering to the subject an effective amount of (i) islet tissue or an islet cell population; and (ii) EGF or a fragment thereof. In embodiments, the islet tissue or islet cell population has been cultured or prepared according to a method disclosed herein. In embodiments, the islet cell population is a cultured islet cell population. In embodiments, the disease is diabetes mellitus. In embodiments, the autoimmune disease is associated with reduced islet cell function or survival. In embodiments, the autoimmune disease is associated with islet cell death. In embodiments, the autoimmune disease is Type 1 diabetes. In embodiments, the subject does not have cancer. In embodiments, the subject does not have a bacterial, fungal, or viral infection.

In an aspect, provided herein is a method of treating Type 1 diabetes in a subject. In embodiments, the method comprises administering to the subject an effective amount of (i) EGF or a fragment thereof, and/or (ii) islet cells.

In embodiments, the increased viability or function of cells in an islet cell population allows for fewer donors to be used. In embodiments, the increased viability or function of cells an islet cell population allows for fewer cells to be used. In embodiments, less than 5000 IEQ/kg of the subject's body weight is administered to the subject. In embodiments, less than 4500 IEQ/kg of the subject's body weight is administered to the subject. In embodiments, less than 4000 IEQ/kg of the subject's body weight is administered to the subject. In embodiments, less than 3500 IEQ/kg of the subject's body weight is administered to the subject. In embodiments, less than 3000 IEQ/kg of the subject's body weight is administered to the subject. In embodiments, less than 2500 IEQ/kg of the subject's body weight is administered to the subject. In embodiments, from 2500 to 5000 IEQ/kg of the subject's body weight is administered to the subject. In embodiments, from 3000 to 5000 IEQ/kg of the subject's body weight is administered to the subject. In embodiments, from 3500 to 5000 IEQ/kg of the subject's body weight is administered to the subject. In embodiments, from 4000 to 5000 IEQ/kg of the subject's body weight is administered to the subject. In embodiments, from 4500 to 5000 IEQ/kg of the subject's body weight is administered to the subject.

In the context of delivering an islet tissue or cell population to a subject “transplanting” and “administering” are used synonymously. An islet tissue or cell population “transplant” is an islet tissue or cell population that is being or has been administered to a subject. In embodiments, an islet cell population is administered by injection or infusion. In embodiments, the injection is an intravenous injection. In embodiments, the islet cell population is administered surgically. In embodiments, the islet cell population is administered into the portal vein of the liver. In embodiments, the administration is via a catheter. In embodiments, placement of the catheter is guided by ultrasound or radiography.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Embodiments

Embodiments include embodiments P1 to P24 following:

Embodiment P1. A method of culturing a plurality of islet cells, the method comprising: culturing the plurality of islet cells in a culture medium comprising epidermal growth factor (EGF) or a fragment thereof, thereby forming a plurality of cultured islet cells.

Embodiment P2. The method of Embodiment P1, wherein the culture medium is an ex vivo culture medium.

Embodiment P3. The method of Embodiment P1 or 2, wherein the culture medium is a liquid culture medium.

Embodiment P4. The method of any one of Embodiments P1 to P3, wherein the plurality of islet cells is obtained from one or more isolated islets.

Embodiment P5. The method of Embodiment P4, wherein the one or more islets are isolated from one or more donor subjects.

Embodiment P6. The method of Embodiment P5, wherein the donor subject is human.

Embodiment P7. The method of any one of Embodiments P1 to P6, wherein the plurality of islet cells is a plurality of fully differentiated islet cells or a plurality of progenitor cells.

Embodiment P8. The method of any one of Embodiments P1 to P6, wherein the plurality of islet cells is a plurality of fully differentiated beta cells, a plurality of fully differentiated alpha cells, a plurality of fully differentiated delta cells, a plurality of progenitor cells, or a mixture thereof.

Embodiment P9. The method of any one of Embodiments P1 to P8, wherein the plurality of islet cells is a plurality of human islet cells.

Embodiment P10. The method of any one of Embodiments P1 to P9, wherein the EGF or a fragment thereof is human EGF or a fragment thereof.

Embodiment P11. The method of Embodiment P10, wherein the human EGF comprises an amino acid sequence of SEQ ID NO: 1.

Embodiment P12. The method of Embodiment P10, wherein the fragment thereof comprises an amino acid sequence of SEQ ID NO: 2 or SEQ ID NO: 3.

Embodiment P13. The method of any one of Embodiments P1 to P12, wherein the EGF or a fragment thereof increases oxygen consumption rate of the islet cells.

Embodiment P14. The method of any one of Embodiments P1 to P13, wherein the EGF or a fragment thereof is present in the culture medium at an amount sufficient to increase oxygen consumption rate of the islet cells.

Embodiment P15. The method of any one of Embodiments P1 to P14, wherein the EGF or a fragment thereof is present in the culture medium at an amount of about 2-10 ng.

Embodiment P16. The method of any one of Embodiments P1 to P15, wherein the cultured plurality of islet cells is more bioactive or has a higher function relative to the plurality of islet cells.

Embodiment P17. The method of any one of Embodiments P1 to P16, further comprising treating diabetes mellitus in a subject in need thereof, the method comprising transplanting to the subject the plurality of cultured islet cells thereby treating the diabetes mellitus.

Embodiment P18. The method of Embodiment P17, wherein the diabetes mellitus is type I diabetes mellitus.

Embodiment P19. An ex vivo culture, comprising a plurality of fully differentiated islet cells in a culture medium, wherein the culture medium comprises epidermal growth factor (EGF) or a fragment thereof.

Embodiment P20. The ex vivo culture of Embodiment P19, wherein the fully differentiated islet cells are fully differentiated beta cells, fully differentiated alpha, fully differentiated delta cells, or a mixture thereof.

Embodiment P21. The ex vivo culture of Embodiment P19 or P20, comprising about 2-10 ng of EGF or a fragment thereof.

Embodiment P22. The ex vivo culture of any one of Embodiments P19 to P21, wherein the EGF or a fragment thereof is human EGF or a fragment thereof.

Embodiment P23. The ex vivo culture of any one of Embodiments P19 to P22, wherein the human EGF comprises an amino acid sequence of SEQ ID NO: 1.

Embodiment P24. The ex vivo culture of any one of Embodiments P19 to P22, wherein the fragment thereof comprises an amino acid sequence of SEQ ID NO: 2 or SEQ ID NO: 3.

Further embodiments include embodiments 1-25 following:

Embodiment 1. A method of increasing the viability or function of an islet cell population, the method comprising culturing the islet cell population in a culture medium comprising an effective amount of epidermal growth factor (EGF) or a fragment thereof for up to 72 hours.

Embodiment 2. The method of Embodiment 1, wherein the islet cell population is cultured in the culture medium for 24 to 72 hours.

Embodiment 3. The method of Embodiment 1 or 2, wherein the effective amount is 2-10 ng/ml.

Embodiment 4. The method of any one of Embodiments 1-3, wherein the culture medium comprises glucose in a concentration of from 3.9 to 5.5 mM.

Embodiment 5. The method of any one of Embodiments 1-4, wherein the islet cell population has been isolated from a subject with a Hemoglobin A1c (HbA1c) greater than 5.6%.

Embodiment 6. The method of any one of Embodiments 1-5, wherein the islet cell population comprises beta cells, alpha cells, delta cells, progenitor cells, or any combination thereof.

Embodiment 7. The method of any one of Embodiments 1-6, wherein the culture medium further comprises gastrin.

Embodiment 8. The method of any one of Embodiments 1-7, wherein the culture medium does not comprise gastrin.

Embodiment 9. The method of any one of Embodiments 1-8, wherein the culture medium further comprises glucagon-like peptide-1 (GLP-1) or a GLP-1 receptor agonist.

Embodiment 10. The method of any one of Embodiments 1-9, wherein the EGF is recombinant EGF.

Embodiment 11. The method of any one of Embodiments 1-10, wherein the EGF is human EGF.

Embodiment 12. The method of any one of Embodiments 1-11, wherein the EGF comprises the amino acid sequence of SEQ ID NO: 1.

Embodiment 13. The method of any one of Embodiments 1-11, wherein the EGF fragment comprises the amino acid sequence of SEQ ID NO: 2 or 3.

Embodiment 14. A method of treating an autoimmune disease in a subject in need thereof, wherein the autoimmune disease is associated with reduced islet cell function or survival, the method comprising administering to the subject an effective amount of (i) EGF or a fragment thereof, and/or (ii) cells from an islet cell population that has been cultured in a culture medium comprising EGF or a fragment thereof for up to 72 hours.

Embodiment 15. The method of Embodiment 14, wherein the autoimmune disease is Type 1 diabetes.

Embodiment 16. The method of Embodiment 14 or 15, wherein the islet cell population has been isolated from a subject with a HbA1c greater than 5.6%.

Embodiment 17. The method of any one of Embodiments claim 14-16, wherein the islet cell population comprises beta cells, alpha cells, delta cells, progenitor cells, or any combination thereof.

Embodiment 18. The method of any one of Embodiments 14-17, wherein the EGF is recombinant EGF.

Embodiment 19. The method of any one of Embodiments 14-18, wherein the EGF comprises the amino acid sequence of SEQ ID NO: 1.

Embodiment 20. The method of any one of Embodiments 14-18, wherein the EGF fragment comprises the amino acid sequence of SEQ ID NO: 2 or 3.

Embodiment 21. The method of any one of Embodiments 14-20, wherein the islet cell population has been isolated from a single donor.

Embodiment 22. The method of any one of Embodiments 14-21, less than 5000 islet equivalent (IEQ) per kg of the subject's body weight is administered to the subject.

Embodiment 23. The method of any one of Embodiments 14-22, comprising administering to the subject an effective amount of cells from an islet cell population that has been cultured in a culture medium comprising EGF or a fragment thereof for up to 72 hours.

Embodiment 24. The method of Embodiment 23, wherein the islet cell population is administered into the portal vein of the subject's liver.

Embodiment 25. An isolated islet cell population that has been cultured in culture medium comprising recombinant EGF for up to 72 hours.

### EXAMPLES

### Example 1

### Epidermal Growth Factor Receptor Immunofluorescence Cell Staining of Human Pancreas tissues.

Tissue sections were fixed in formalin and embedded in paraffin blocks according to standard procedures. 4-6-micron thick tissue sections were prepared using a microtome, and applied to electrostatically-charged micro slides. These were deparaffinized in xylenes using three changes for 5 minutes each. Then, sections were hydrate gradually through graded alcohols. Finally, washed in deionized H₂O for 1 minute with stirring and aspirated the excess liquid from slides. Alternatively, Frozen Tissue Sections and Tissue Culture Cells can be used. Antigen unmasking was performed by standard methods. Direct and indirect immunofluorescence staining of cells or tissue sections, were performed using the standard methods with conventional secondary antibodies commercially available. Specimens were incubated for 30 minutes with 10% normal blocking serum in PBS or blocking reagents, and then washed with three changes of PBS for 5 minutes each. Primary antibodies were incubated for 60 minutes to overnight at room temperature or overnight at 4° C. Specimens were washed with three changes of PBS for 5 minutes each and incubated at room temperature for 60 minutes, in a dark chamber, with either a fluorophore-conjugated secondary antibody diluted in either PBS with 1.5% normal blocking serum, or blocking reagents or a fluorophore-conjugated antibody. Then, cover slips were mounted with either an aqueous, or a hard-set mounting medium. Slides were examined using a fluorescence microscope (Keyence) with appropriate filters. The antibodies used included: EGFR Antibody (A-10) PE (Santa Cruz Biotechnology sc-373746 PE) and rabbit anti-insulin AN735-5M (Biogenex).

### Example 2

### Glucose-Stimulated Increment in Oxygen Consumption Rate (OCR) Test Using Seahorse Analyzers.

Protocol for islet glucose stimulation test with EGF is provided below.

Materials:
Islets
EGF stock: lug/mL
CMRL media
50mL tubes
6-well plates or T25/T75 flasks (depending on # islets)
Seahorse calibration solution
24-well Seahorse assay plate

Prior to assay islets are cultured in islet capture media and stored in incubator.

Day 1:
1. Take islets in T75 flask from incubator
2. Slowly take the islets up with serological pipet and resuspend in 50ml tube
3. Wait 5 min till islets have fallen to the bottom of the tube
4. In the meantime, prepare 5mL of CMRL media in well/condition in 6 well plate
5. Discard media with serological pipet
6. Wash with fresh CMRL media
7. Wait 5 min till islets have fallen to the bottom of the tube
8. In the meantime, prepare EGF conditions in another 6-well plate(or T25/T75 flask):
   Aliquot 5 mL of CMRL media in one well/condition
   add 0 EGF for control condition
      10 uL for the 2 ng/mL condition
      50 uL for the 10 ng/mL condition
9. With a 1mL pipet, take up pellet of islets on the bottom of the tube and transfer to wells in the 6-well plate
10. Make sure there are equal amounts of islets per condition per well
11. Swirl 6-well plate to center the islets
12. Transfer islets to 6 well plate with EGF conditions
13. Incubate cells for 24 hrs in incubator
14. Calibrate Seahorse assay plate with calibration solution
15. Store in —CO₂ incubator overnight

Materials:
Islets
XF Seahorse base media
Glucose
Oligomycin
24 well islet capture plate
Sharp razor
Petri dish
Capture screens
Insertion tool
Pincet
Black screen
MA media the formula of which is provided below

MA media (3 mM Glucose)

| | | |
|---|---|---|
| ml | 60 | 50 |
| 2.8M Glucose (ul) | 0.064 | 0.054 |
| FBS | 0.600 | 0.500 |
| MA base media | 59.34 | 49.45 |
| | 60.00 | 50.00 |
| pH to 7.4: 1M NaOH | | |
| ul | 36 | 30 |

Day 2:
1. Prepare low glucose media (see table above) and put in —CO₂ incubator
2. Put 470-500 uL low glucose media/well in 24 well islet capture plate
3. Put media in petri dish and soak capture screens in there
4. Cut off tip of 10 uL pipet with sharp sterile razor
5. Swirl the islets to the center of 6 well plate under black screen
6. Take islets from 6-well plate with a 10 uL pipet and transfer to low glucose media 24 well plate. Capture 5 uL islets/well at a time and resuspend in islet capture well into 3 mm circle
7. Look under microscope whether all islets are in capture well, otherwise shove them in there with a pipet tip

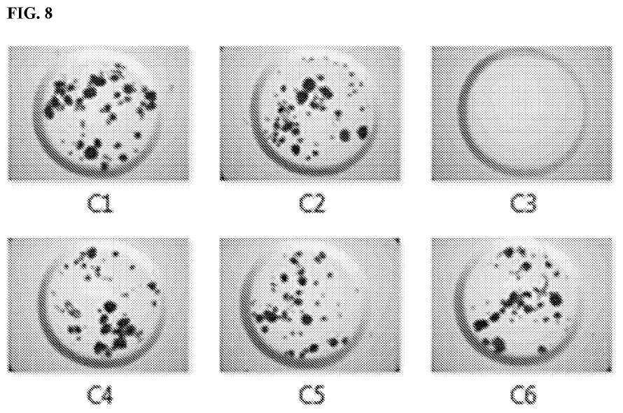

See FIG. 8.
8. Put rimside of capture screens up with a pincet under the microscope
9. Put capture screens in every well with insertion tool
10. Check whether islets are under capture screen
11. Incubate for at least 3 hours in incubator —CO2
12. In the meantime(after 1 hour), prepare compounds:
   20mM glucose
   5 uM oligomycin in low glucose media

| Direct- [symbol] 3 mM Injection, followed by 20 mM, [symbol] [symbol] | | | | | | | | | | | | | |
|---|---|---|---|---|---|---|---|---|---|---|---|---|---|
| | | | Want | | | | Injection concentration needed | | | ul Stock | 3 mM glucose | | |
| Have | | | | | | moles | moles | [symbol] | | Glucose [symbol] | MA Media (ml) | | half |
| [symbol] | vol. | moles | [symbol] | vol. | moles | correction | needed | vol [symbol] | mM | 2.8 | [symbol] | check | vol. |
| [symbol] | 0. [symbol] 00 | [symbol] .50 | 20 | 0.850 | 1 [symbol] .00 | 0.22 [symbol] | 11.2 [symbol] | 0.075 | 150 | [symbol] | 1514 | 1500 | 757 |

| Injection volume: 75 ul of 3 mM glucose correction | | | | |
|---|---|---|---|---|
| [symbol] | vol | moles | | |
| [symbol] | 0.075 | 0.275 | make 1.8 ml for [symbol] plate | 1.8 ml |
| | | | check | 17.35 mM |

| | [symbol] STOCKS | [HAVE] | [WANT] | ENTER | | | | |
|---|---|---|---|---|---|---|---|---|
| Injection | 10 X Injection | DMSO | 10 X Injection | Total MA | [symbol] | MA | half | |
| Port | Compound (want) | Stock [mM] | Compound [uM] | media (ml) | Compounds | Media (ul) | vol. | |
| [symbol] | Oligomycin [symbol] uM [symbol] | [symbol] 0 | [symbol] 0 | 1.7 | 1.7 | 1698 | 849 | make 1.7 ml for a [symbol] plate of 75 ul |

[symbol]

indicates data missing or illegible when filed

13. Load 75 uL/drug injection port:
   media in background wells
   A: 3 mM low glucose
   B: 20 mM high glucose
   C: Oligomycin
14. Keep plate in —CO2 incubator for 1 hour
15. Last 20 min: calibrate Seahorse assay plate
16. Set up plate in Wave software
17. Run assay on Seahorse analyzer

### Example 3

### Evidence Indicating the Expression of the EGF Receptor (EGFR) in Human Pancreatic Islets.

1) The EGF receptor (EGFR) is expressed in human pancreatic islets.

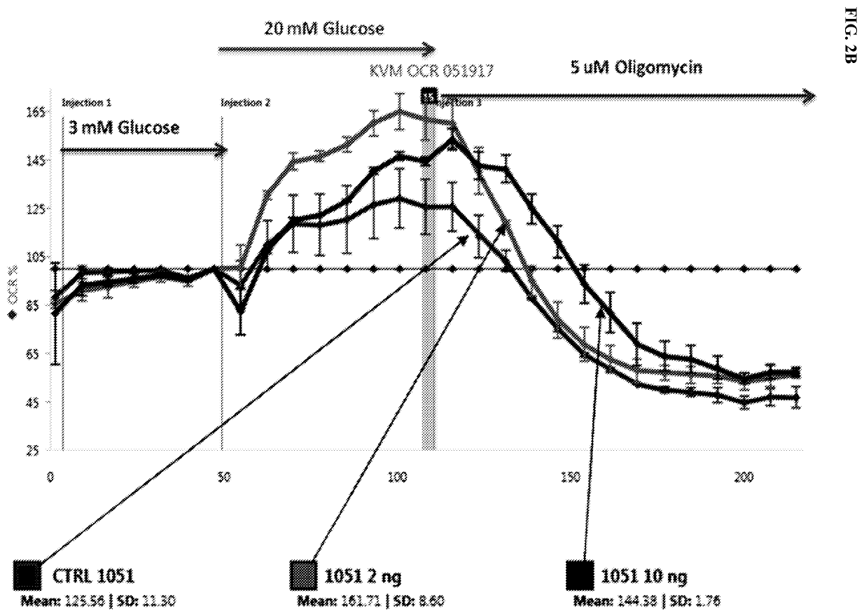

Consistent evidence has been obtained for the expression of the EGF receptor (EGFR) in human pancreatic tissues from non-diabetic and hyperglycemic donors, evaluated by immunohistochemistry using standard procedures and detection systems. Detection of both the extracellular and intracellular domains (ECD and ICD) of EGFR was obtained. FIG. 3 displays representative images of paraffin-embedded pancreatic tissue sections from a normal donor stained for EGFR-ECD and EGFR-ICD. Original magnification is 40×, taken on a Zeiss LSM 700 Confocal microscope.

2) Recombinant EGF added on isolated human islets under stress conditions has a protective effect.

Islet cytotoxicity was analyzed using approximately 70 islets/well isolated human pancreatic islets cultured in Seahorse XF24 islet-capture-microplates at 37° C. and 5% CO₂ in 200 μl of either in CMRL islet culture media with and without pro-inflammatory cytokines in triplicates, i.e.: Media (Control), high concentration (High Cyto) of human recombinant cytokines (1000 IU/ml IFN-γ, 1000 IU/ml TNF-α and 50 IU/ml IL-1β) or low concentrations of human recombinant cytokines (Low Cyto: 100 IU/ml IFN-γ, 100 IU/ml TNF-α and 5 IU/ml IL-1β). Additional conditions included, supernatant (100%) of a β-cell antigen specific autoreactive T-cell clone isolated from a prediabetic donor and stimulated in vitro with islet peptide (T-cell SP) or a control peptide (T-cell NS). Islets were cultured with or without 10 ng/ml of human recombinant EGF in all culture conditions. Cell death was measured after 24 h by incubating 2 μg/ml of Propidium Iodide (PI) for 15 min at room temperature, and scanned in the Celigo Image Cytometer. Two-way ANOVA, *p<0.05, T-cell SP p=0.0231, High Cyto p=0.0137, n=3.

The EGF protective effect is observed in the range from 0.001 to 10 ng/ml.

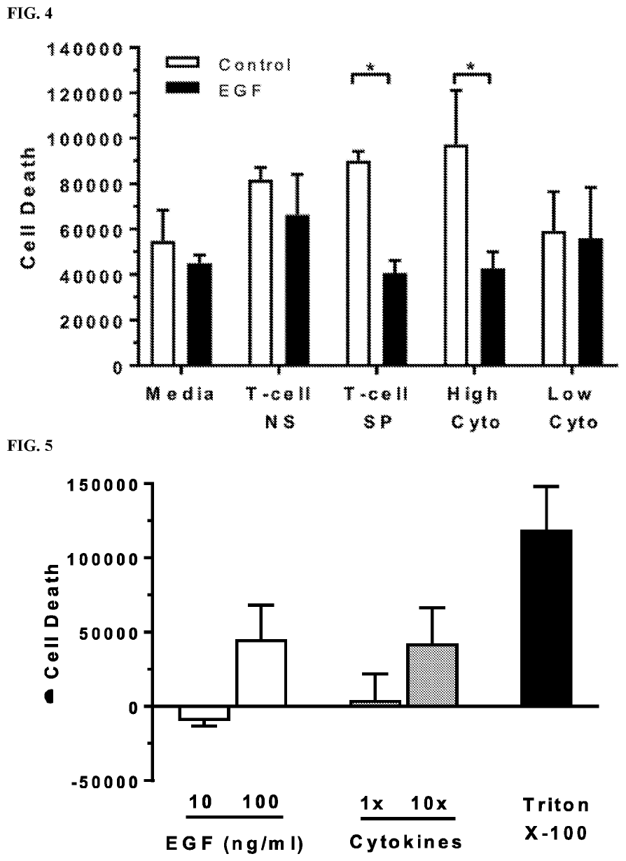

Additionally, it was found that exogenous EGF might reduce the immunogenicity of normal human islets for transplantation by reducing the HLA class I expression. FIG. 6 shows a western blot of protein extracts from human islets from a normal donor treated with or without 10 ng/ml EGF and 50 units/mL IL-1β, 1,000 units/mL TNF-α, and 1,000 units/mL IFN-γ for 24 h.

What is claimed is:

1. A method of increasing the viability or function of an islet cell population, the method comprising culturing the islet cell population in a culture medium comprising an effective amount of epidermal growth factor (EGF) or a fragment thereof for up to 72 hours.

2. The method of claim 1, wherein the islet cell population is cultured in the culture medium for 24 to 72 hours.

3. The method of claim 1, wherein the effective amount is 2-10 ng/ml.

4. The method of claim 1, wherein the culture medium comprises glucose in a concentration of from 3.9 to 5.5 mM.

5. The method of claim 1, wherein the islet cell population has been isolated from a subject with a Hemoglobin A1c (HbA1c) greater than 5.6%.

6. The method of claim 1, wherein the islet cell population comprises beta cells, alpha cells, delta cells, progenitor cells, or any combination thereof.

7. The method of claim 1, wherein the culture medium further comprises gastrin.

8. The method of claim 1, wherein the culture medium does not comprise gastrin.

9. The method of claim 1, wherein the culture medium further comprises glucagon-like peptide-1 (GLP-1) or a GLP-1 receptor agonist.

10. The method of claim 1, wherein the EGF is recombinant EGF.

11. The method of claim 1, wherein the EGF is human EGF.

12. The method of claim 11, wherein the EGF comprises the amino acid sequence of SEQ ID NO: 1.

13. The method of claim 1, wherein the EGF fragment comprises the amino acid sequence of SEQ ID NO: 2 or 3.

14. A method of treating an autoimmune disease in a subject in need thereof, wherein the autoimmune disease is associated with reduced islet cell function or survival, the method comprising administering to the subject an effective amount of (i) EGF or a fragment thereof, and/or (ii) cells from an islet cell population that has been cultured in a culture medium comprising EGF or a fragment thereof for up to 72 hours.

15. The method of claim 14, wherein the autoimmune disease is Type 1 diabetes.

16. The method of claim 14, wherein the islet cell population has been isolated from a subject with a HbA1c greater than 5.6%.

17. The method of claim 14, wherein the islet cell population comprises beta cells, alpha cells, delta cells, progenitor cells, or any combination thereof.

18. The method of claim 14, wherein the EGF is recombinant EGF.

19. The method of claim 14, wherein the EGF comprises the amino acid sequence of SEQ ID NO: 1.

20. The method of claim 14, wherein the EGF fragment comprises the amino acid sequence of SEQ ID NO: 2 or 3.

21. The method of claim 14, wherein the islet cell population has been isolated from a single donor.

22. The method of claim 14, less than 5000 islet equivalent (IEQ) per kg of the subject's body weight is administered to the subject.

23. The method of claim 14, comprising administering to the subject an effective amount of cells from an islet cell population that has been cultured in a culture medium comprising EGF or a fragment thereof for up to 72 hours.

24. The method of claim 14, wherein the islet cell population is administered into the portal vein of the subject's liver.

25. An isolated islet cell population that has been cultured in culture medium comprising recombinant EGF for up to 72 hours.
